(19) United States
(12) Patent Application Publication
(10) Pub. No.: US 2024/0382515 A1
(43) Pub. Date: Nov. 21, 2024

(54) WATER-SOLUBLE DIETARY FIBER, USE THEREOF IN PREPARATION OF MEDICAMENT THAT PROMOTES METABOLISM OF ELLAGIC ACID INTO UROLITHIN A, AND PHARMACEUTICAL COMPOSITION

(71) Applicant: ZHEJIANG UNIVERSITY, Hangzhou City (CN)

(72) Inventors: Shiguo CHEN, Hangzhou City (CN); Laiming ZHANG, Hangzhou City (CN); Haibo PAN, Hangzhou City (CN); Xingqian YE, Hangzhou City (CN); Donghong LIU, Hangzhou City (CN); Tian DING, Hangzhou City (CN)

(21) Appl. No.: 18/214,162

(22) Filed: Jun. 26, 2023

(30) Foreign Application Priority Data
May 19, 2023 (CN) 2023105790496

(51) Int. Cl. A61K 31/715; A61P 3/00

(52) U.S. Cl. CPC A61K 31/715; A61P 3/00

(57) ABSTRACT

The present disclosure provides a water-soluble dietary fiber (WSDF), use thereof in preparation of a medicament that promotes metabolism of ellagic acid (EA) into urolithin A, and a pharmaceutical composition, and belongs to the technical field of biological pharmaceuticals. The WSDF provided by the present disclosure includes at least two components selected from the group consisting of gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin. In the present disclosure, compounding the foregoing different combinations of the WSDF and EA to feed mice may significantly increase the average urolithin A content in mouse feces. Visibly, the WSDF alone or in combination with EA may improve metabotypes of the EA in the body and increase urolithin A content, providing a new means for treating diabetes, obesity, senescence and central nervous system lesions.

### CROSS REFERENCE TO RELATED APPLICATION

This patent application claims the benefit and priority of Chinese Patent Application No. 2023105790496 filed with the China National Intellectual Property Administration on May 19, 2023. the disclosure of which is incorporated by reference herein in its entirety as part of the present application.

### TECHNICAL FIELD

The present disclosure belongs to the technical field of biological pharmaceuticals, and particularly relates to a water-soluble dietary fiber (WSDF), use thereof in preparation of a medicament that promotes metabolism of ellagic acid (EA) into urolithin A, and a pharmaceutical composition.

### BACKGROUND

Ellagic acid (EA) is a widely sourced and easily extracted natural polyphenol present in berries (pomegranate, strawberry, etc.) and nuts (walnut, chestnut, etc.). In the gastrointestinal tract, ellagitannin is hydrolyzed to release hexahydroxy diphenyl acid and rapidly condensed into EA. EA is a strong polar molecule that is hardly absorbed and used by the gastrointestinal tract. Therefore, EA cannot exert effective bioactivity due to low bioavailability thereof. Specific microflora in the human intestinal tract can effectively metabolize EA and produce a range of urolithins, which can be effectively used and produce a range of probiotic activity compared with EA. Japanese patent JP2022190124 indicates that urolithin A exerts probiotic activity in mouse models of diabetes, obesity, senescence, and central nervous system lesions. Therefore. EA as a precursor of urolithin A is easily extracted due to a wealth of sources and attract widespread attention in the field of food.

Current research has shown that EA can be metabolized into different urolithin derivatives in the colon. Herein, urolithin A, isourolithin A and urolithin B are metabolic end products of EA. The metabotype of EA in population can be divided into metabotypes A, B, and 0 according to the differences in metabolic end products of EA in different populations; metabotype A population finally produce urolithin A, metabotype B population produce urolithin A, urolithin B and isourolithin A, and metabotype 0 population cannot metabolize EA to produce urolithins (Tomás-Barberán F A, Garcia-Villalba R, Gonzalez-Sarrias A, et al. EA metabolism by human gut microbiota: consistent observation of three urolithin metabotypes in intervention trials, independent of food source, age, and health status [J]. Journal of Agricultural and Food Chemistry, 2014, 62 (28): 6535-6538.). Therefore, in metabotype 0 population, EA cannot be metabolized into urolithin A that produces effective probiotic activity; in metabotype B population, the yield of urolithin A decreases and cannot reach an effective dose. This leads to limited use of EA in metabotypes 0 and B populations. The difference in metabolic end product among populations is mainly related to the difference in human gut microflora composition. Therefore, it is urgent to develop a method for improving metabolic transformation of EA in metabotypes 0 and B populations.

Compounding with polysaccharides can effectively improve the bioactivity of EA: Chinese patent CN113812629 discloses a method of preparing a starch-EA inclusion complex and use thereof in antioxidant foods. The complex is formed by grinding EA and starch at high speed, and shows excellent slow release performance and antioxidant activity in vitro. Chinese patent CN115191540 discloses a composition containing EA and a method of preparation and use thereof. The composition is prepared with chitooligosaccharide, inulin and EA, and can effectively inhibit the growth of gastric and intestine cancer cells. Chinese patent CN111107908 discloses a composition comprising EA compound, which is prepared by compounding various glycerophospholipids and metal oxides with EA. The composition increases the antioxidant activity of EA and the overall stability of the composition. However, there is no report of compounding polysaccharides with EA to promote EA metabolism.

Dietary fiber is a well-recognized human health nutrient. Previous research has shown that dietary fiber is of importance to gastrointestinal health by regulating intestinal microflora. In addition, mechanism research has indicated that physiological functions of different dietary fibers largely depend on physicochemical properties thereof, one of which is solubility. Compared with insoluble dietary fibers, soluble dietary fibers (SDFs) are easily acquired and metabolized by fiber-degrading microbes in the gut, which produces a range of probiotic and functional metabolites. Given the poor effect of in vivo metabolism of EA into urolithin A, there is no report of dietary fiber capable of improving the metabolism of EA into urolithin A so far.

### SUMMARY

In view of this, the present disclosure aims to provide a WSDF. Intestinal microflora structure is improved by means of different SDF combinations, thereby promoting efficiency of metabolism of EA into urolithin A.

The present disclosure provides a WSDF, including at least two components selected from the group consisting of gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin.

In some embodiments, the WSDF includes at least one selected from the group consisting of a first WSDF formed by gellan gum, guar gum and carrageenan, a second WSDF formed by fructooligosaccharide, xylooligosaccharide and malto-oligosaccharide, a third WSDF formed by pectin, β-glucan and polydextrose, a fourth WSDF formed by sodium alginate, gum arabic and resistant dextrin, and a fifth WSDF formed by xanthan gum, tragacanth gum and inulin; and

a mass ratio of three components within the first WSDF, the second WSDF, the third WSDF, the fourth WSDF or the fifth WSDF is (1-3):(1-3):(1-3).

The present disclosure provides a pharmaceutical composition for promotion of metabolism of EA into urolithin A, including the WSDF and EA.

The WSDF and the EA have a mass ratio of (100-250):3.

In some embodiments, the WSDF and EA have a mass ratio of 250:3.

In some embodiments, EA has a structural formula represented by formula I:

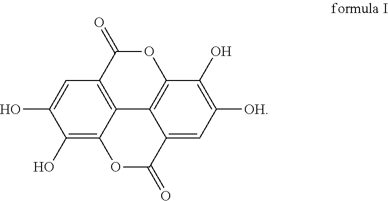 [SMILES: O=c1oc2c(O)c(O)cc3c(=O)oc4c(O)c(O)cc1c4c23]

The present disclosure provides use of the WSDF or the pharmaceutical composition in preparation of a medicament that promotes metabolism of EA into urolithin A.

The present disclosure provides a product that promotes metabolism of EA into urolithin A. The WSDF or the pharmaceutical composition is used as an active ingredient, and excipients are further included.

In some embodiments, the product that promotes metabolism of EA into urolithin A includes at least one selected from the group consisting of a medicament, a health care product and a health food.

The present disclosure provides use of the WSDF or the pharmaceutical composition in preparation of a medicament for prophylaxis and/or treatment of at least one disease selected from the group consisting of diabetes, obesity, senescence, and central nervous system lesions.

In some embodiments, patients with the at least one disease belong to one selected from the group consisting of metabotypes A, B and 0 populations of EA metabolism.

The present disclosure provides a WSDF, including at least two components selected from the group consisting of gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin. In the present disclosure, a plurality of components for influencing metabotypes of EA are obtained by selection and optimization of a plurality of WSDFs. In the present disclosure, mice are fed with combinations of different WSDFs with EA and monitored for EA metabolism. It is indicated that average urolithin A content in mouse feces is increased from 41.0-32.3 μg/g of feces (control group, fed with EA alone) to 41.4-87.4 μg/g of feces. Visibly, combinations of different WSDFs are conducive to increasing the level of metabolism of EA into urolithin A in the body, providing a new means for treating a plurality of diseases with urolithin A and being of significant importance to clinical disease treatment.

Further, the present disclosure specifically limits several schemes of WSDF combinations. Combinations of different WSDFs have different effects on metabotypes of EA. Herein, a fifth WSDF formed by xanthan gum, tragacanth gum and inulin has an optimal effect on the improvement of metabolism of the EA into urolithin A, followed by a second WSDF formed by fructooligosaccharide, xylooligosaccharide and malto-oligosaccharide.

### BRIEF DESCRIPTION OF THE DRAWINGS

FIG. 1 illustrates selection results of WSDFs that promotes metabolism of EA into urolithin A; and

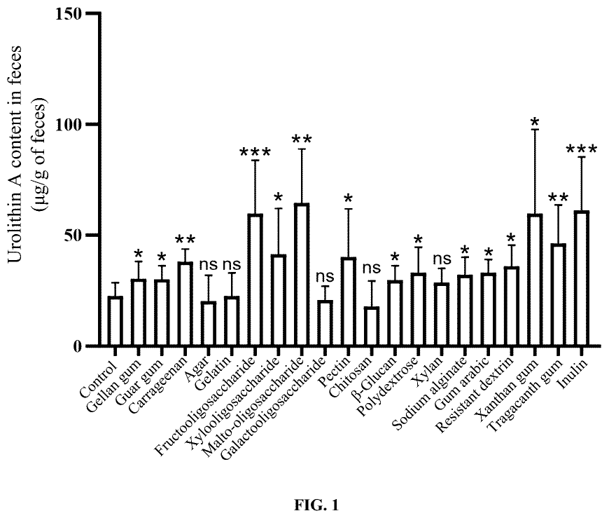

FIG. 2 illustrates changes in content of urolithin A metabolized from EA by different WSDF combinations in Examples 2, 3, 4, 5, and 6, where *P<0.05, **P<0.01. ***P<0.001 and ****P<0.0001 versus control group.

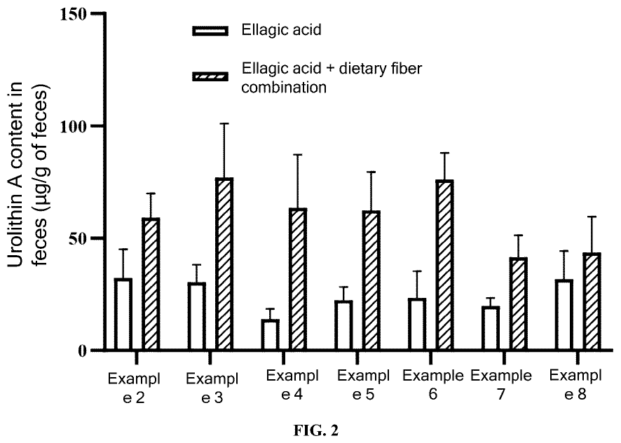

### DETAILED DESCRIPTION OF THE EMBODIMENTS

The present disclosure provides a WSDF, including at least two components selected from the group consisting of gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin.

In the present disclosure, the WSDF preferably includes at least one selected from the group consisting of a first WSDF formed by gellan gum, guar gum and carrageenan, a second WSDF formed by fructooligosaccharide, xylooligosaccharide and malto-oligosaccharide, a third WSDF formed by pectin, β-glucan and polydextrose, a fourth WSDF formed by sodium alginate, gum arabic and resistant dextrin, and a fifth WSDF formed by xanthan gum, tragacanth gum and inulin, more preferably the second WSDF and the fifth WSDF, and most preferably the fifth WSDF. A mass ratio of three components within the first WSDF, the second WSDF, the third WSDF, the fourth WSDF or the fifth WSDF is preferably (1-3):(1-3):(1-3), and more preferably 1:1:1. In the present disclosure, there is no particular limitation on sources of the foregoing WSDFs, as long as the sources of the WSDFs known to those skilled in the art are used. In an embodiment of the present disclosure, the gellan gum, the guar gum, the carrageenan, the fructooligosaccharide, the xylooligosaccharide, the malto-oligosaccharide, the pectin, the β-glucan, the polydextrose, the sodium alginate, the gum arabic, the resistant dextrin, the xanthan gum, the tragacanth gum, and the inulin are purchased from Sigma.

In an embodiment of the present disclosure, it is found in selection from of a plurality of WSDFs that different WSDF combinations may significantly influence the efficiency of metabolism of EA into urolithin A. For example, 20 dietary fibers including gellan gum, guar gum, carrageenan, agar, gelatin, fructooligosaccharide, xylooligosaccharide, malto-oligosaccharide, galactooligosaccharide, pectin, chitosan, β-glucan, polydextrose, xylan, sodium alginate, gum arabic, resistant dextrin, xanthan gum, tragacanth gum, and inulin are selected by an animal experiment. It is found that 15 dietary fibers can promote EA metabolism in mice, namely gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin.

The present disclosure provides a pharmaceutical composition that promotes metabolism of EA into urolithin A, including the WSDF and EA. The WSDF and the EA have a mass ratio of (100-250):3. The WSDF and the EA preferably have a mass ratio of 250:3.

In the pharmaceutical composition according to the present disclosure, the WSDF improves the efficiency of the metabolism of EA into urolithin A by improving a structure of intestinal probiotics in the body. EA is present as a metabolic substrate. EA may be extracted from one or more selected from the group consisting of pomegranate, blackberry, strawberry, pomegranate, wolfberry, raspberry, white oak acorn, cranberry, and pecan. EA preferably has a structure represented by formula I and is purchased from Sigma.

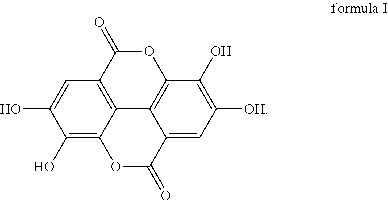 [SMILES: O=c1oc2c(O)c(O)cc3c(=O)oc4c(O)c(O)cc1c4c23]

The present disclosure provides use of the WSDF or the pharmaceutical composition in preparation of a medicament that promotes metabolism of EA into urolithin A.

According to results of examples of the present disclosure, mice are fed with a combination of gellan gum, guar gum and carrageenan with EA and monitored for EA metabolism. It is indicated that the average content of urolithin A in mouse feces is increased from 31.9 μg/g of feces to 59.1 μg/g of feces; and when the mice are fed with a combination of pectin, β-glucan and polydextrose with EA, the average content of the urolithin A in mouse feces is increased from 31.9 μg/g of feces to 63.5 μg/g of feces. When the mice are fed with a combination of sodium alginate, gum arabic and resistant dextrin with EA, the average content of urolithin A in mouse feces is increased from 31.9 μg/g of feces to 62.4 μg/g of feces. When the mice are fed with a combination of fructooligosaccharide, xylooligosaccharide and malto-oligosaccharide with EA, the average content of urolithin A in mouse feces is increased from 31.9 μg/g of feces to 77.1 μg/g of feces. When the mice are fed with a combination of xanthan gum, tragacanth gum, and inulin with EA, the average content of urolithin A in mouse feces is increased from 31.9 μg/g of feces to 87.4 μg/g of feces.

The present disclosure provides a product that promotes metabolism of EA into urolithin A. The WSDF or the pharmaceutical composition is used as an active ingredient, and excipients are further included.

In the present disclosure, preferably, the product that promotes metabolism of EA into urolithin A includes at least one selected from the group consisting of a medicament, a health care product and a health food. In an embodiment of the present disclosure, the pharmaceutical composition is added as an additive to an animal feed, so that a medicated feed capable of promoting the metabolism of EA into urolithin A in mice is prepared. In the present disclosure, the medicament is preferably administered to a population at a daily dose of no less than 5 mg EA/kg of body weight. EA is administered to a mouse at a daily dose of 60 mg/kg of body weight. The WSDF is administered to the mouse at a daily dose of 5,000 mg/kg of body weight.

In view of a fact that urolithin A could reach the objective of treating a plurality of diseases, the present disclosure provides use of the WSDF or the pharmaceutical composition in preparation of a medicament for prophylaxis and/or treatment of at least one disease selected from the group consisting of diabetes, obesity, senescence, and central nervous system lesions.

In the present disclosure, patients with the at least one disease preferably belong to one selected from the group consisting of metabotypes A, B and 0 populations of EA metabolism.

In the present disclosure, there is no particular limitation on dosage forms and preparation methods of the medicament, as long as a dosage form and a preparation method known in the art are used.

The WSDF, the use thereof in preparation of a medicament that promotes metabolism of EA into urolithin A, and the pharmaceutical composition provided by the present disclosure will be described in detail below with reference to examples, but they should not be construed as limiting the claimed scope of the present disclosure.

### EXAMPLE 1

### Method for Optimizing and Screening Components Capable of Promoting Metabolism of EA Into Urolithin A From a Plurality of WSDFs

One hundred and sixty-eight specific-pathogen-free (SPF)-grade metabotype A C57BL/6 mice were selected. Ain93G purified diet was used as a basal diet. The addition proportion of EA was 0.06%, and that of gellan gum, guar gum, carrageenan, agar, gelatin, fructooligosaccharide, xylooligosaccharide, malto-oligosaccharide, galactooligosaccharide, pectin, chitosan, β-glucan, polydextrose, xylan, sodium alginate, gum arabic, resistant dextrin, xanthan gum, tragacanth gum, and inulin was 5%. The mice were randomized into 21 groups (n=8). The control group was fed with Ain93G diet composed of EA alone for seven days. The remaining groups were each fed with a mixed diet composed of EA and one of the foregoing dietary fibers for seven days. Mouse feces were collected on the last day of the experiment, and the mice were monitored for EA metabolism.

It was indicated that there were 20 dietary fibers capable of effectively promoting the EA metabolism in mice, namely gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin.

### EXAMPLE 2

### Combination of Gellan Gum, Guar Gum and Carrageenan With EA

Forty SPF-grade C57BL/6 mice were selected. Ain93G purified diet was used as a basal diet. The addition proportion of EA was 0.06%, and that of gellan gum, guar gum and carrageenan was 5%. An Ain93G diet composed of EA alone was fed daily within the first seven days, while a diet composed of gellan gum, guar gum and carrageenan (in a mass ratio of 1:1:1) was fed daily within the last seven days. Mouse feces were collected every seven days during the experiment, and the mice were monitored for EA metabolism. The mouse feces were added to dimethyl sulfoxide (DMSO) in a volume-mass ratio of 1:10, fully ground in a grinder, and centrifuged at 8,000 rpm for 10 min at 4° C.; the resulting supernatant was filtered through a 0.22 μm organic membrane, followed by high performance liquid chromatography (HPLC). The HPLC of urolithin A was implemented as follows:

The liquid chromatograph was Waters e2695 HPLC System; the detector was Waters 2489 UV/Vis Detector; the detection wavelength was 305 nm; the chromatographic column was Eclipse XDB-C18 Column (250×4.6 mm, 5.0 μm; Agilent); the column temperature was 30° C.; the sample size was 10 μL; and the elution speed was 1.0 mL/min.

The mobile phases were 0.5% formic acid in water (A) and acetonitrile (B). Namely, A was composed of 0.5% formic acid and 99.5% double distilled water, while B was composed of 100% acetonitrile, where % represents volume %.

The elution gradient was programmed as follows: 0-20 min, 5%-25% B; 20-25 min, 25%-70% B; 25-26 min, 70%-5% B; and 26-35 min, 5% B.

It was indicated that the average content of urolithin A in mouse feces was increased from 32.3 μg/g of feces to 59.1 μg/g of feces (P<0.05).

### EXAMPLE 3

### Combination of Fructooligosaccharide, Xylooligosaccharide and Malto-Oligosaccharide With EA

Forty SPF-grade C57BL/6 mice were selected. Ain93G purified diet was used as a basal diet. The addition proportion of EA was 0.06%, and that of fructooligosaccharide, xylooligosaccharide and malto-oligosaccharide was 5%. An Ain93G diet composed of EA alone was fed daily within the first seven days, while a mixed diet composed of fructooligosaccharide, xylooligosaccharide and malto-oligosaccharide (in a mass ratio of 1:1:1) with EA was fed daily within the last seven days. Mouse feces were collected every seven days during the experiment, and the mice were monitored for EA metabolism. The HPLC of urolithin A was the same as that in Example 2.

It was indicated that the average content of the urolithin A in mouse feces was increased from 30.3 μg/g of feces to 77.1 μg/g of feces (P<0.0001).

### EXAMPLE 4

### Combination of Pectin, β-Glucan and Polydextrose With EA

Forty SPF-grade C57BL/6 mice were selected. Ain93G purified diet was used as a basal diet. The addition proportion of EA was 0.06%, and that of pectin, β-glucan and polydextrose was 5%. An Ain93G diet composed of EA alone was fed daily within the first seven days, while a mixed diet composed of pectin, β-glucan and polydextrose (in a mass ratio of 1:1:1) with EA was fed daily within the last seven days. Mouse feces were collected every seven days during the experiment, and the mice were monitored for EA metabolism. The HPLC of urolithin A was the same as that in Example 2.

It was indicated that the average content of the urolithin A in mouse feces was increased from 14.0 μg/g of feces to 63.5 μg/g of feces (P<0.01).

### EXAMPLE 5

### A Combination of Sodium Alginate, Gum Arabic and Resistant Dextrin With EA

Forty SPF-grade C57BL/6 mice were selected. Ain93G purified diet was used as a basal diet. The addition proportion of EA was 0.06%, and that of sodium alginate, gum arabic and resistant dextrin was 5%. An Ain93G diet composed of EA alone was fed daily within the first seven days, while a mixed diet composed of sodium alginate, gum arabic and resistant dextrin (in a mass ratio of 1:1:1) with EA was fed daily within the last seven days. Mouse feces were collected every seven days during the experiment, and the mice were monitored for EA metabolism. The HPLC of urolithin A was the same as that in Example 2.

It was indicated that the average content of the urolithin A in mouse feces was increased from 22.5 μg/g of feces to 62.4 μg/g of feces (P<0.05).

### EXAMPLE 6

### Combination of Xanthan Gum, Tragacanth Gum and Inulin With EA

Forty SPF-grade C57BL/6 mice were selected. Ain93G purified diet was used as a basal diet. The addition proportion of EA was 0.06%, and that of xanthan gum, tragacanth gum and inulin was 5%. An Ain93G diet composed of EA alone was fed daily within the first seven days, while a mixed diet composed of xanthan gum, tragacanth gum and inulin (in a mass ratio of 1:1:1) with EA was fed daily within the last seven days. Mouse feces were collected every seven days during the experiment, and the mice were monitored for EA metabolism. The HPLC of urolithin A was the same as that in Example 2.

It was indicated that the average content of the urolithin A in mouse feces was increased from 23.4 μg/g of feces to 87.4 μg/g of feces (P<0.05).

### EXAMPLE 7

### Combination of Inulin, Polydextrose and Sodium Alginate With EA

Forty SPF-grade C57BL/6 mice were selected. Ain93G purified diet was used as a basal diet. The addition proportion of EA was 0.06%, and that of inulin, polydextrose and sodium alginate was 5%. An Ain93G diet composed of EA alone was fed daily within the first seven days, while a mixed diet composed of inulin, polydextrose and sodium alginate (in a mass ratio of 1:1:1) with EA was fed daily within the last seven days. Mouse feces were collected every seven days during the experiment, and the mice were monitored for EA metabolism. The HPLC of urolithin A was the same as that in Example 2.

It was indicated that the average content of the urolithin A in mouse feces was increased from 19.8 μg/g of feces to 41.4 μg/g of feces (P<0.05).

### EXAMPLE 8

### Combination of Carrageenan, Fructooligosaccharide and Pectin With EA

Forty SPF-grade C57BL/6 mice were selected. Ain93G purified diet was used as a basal diet. The addition proportion of EA was 0.06%, and that of carrageenan, fructooligosaccharide and pectin was 5%. An Ain93G diet composed of EA alone was fed daily within the first seven days, while a mixed diet composed of carrageenan, fructooligosaccharide and pectin (in a mass ratio of 1:1:1) with EA was fed daily within the last seven days. Mouse feces were collected every seven days during the experiment, and the mice were monitored for EA metabolism. The HPLC of urolithin A was the same as that in Example 2.

It was indicated that the average content of the urolithin A in mouse feces was increased from 31.9 μg/g of feces to 43.7 μg/g of feces (P<0.05).

The above descriptions are merely preferred embodiments of the present disclosure. It should be noted that a person of ordinary skill in the art may further make several improvements and modifications without departing from the principle of the present invention, but such improvements and modifications should be deemed as falling within the protection scope of the present invention.

What is claimed is:

1. A water-soluble dietary fiber (WSDF), comprising at least two components selected from the group consisting of gellan gum, guar gum, carrageenan, fructooligosaccharide, xylooligosaccharide, malto-oligosaccharide, pectin, β-glucan, polydextrose, sodium alginate, gum arabic, resistant dextrin, xanthan gum, tragacanth gum, and inulin.

2. The WSDF according to claim 1, comprising at least one selected from the group consisting of a first WSDF formed by gellan gum, guar gum and carrageenan, a second WSDF formed by fructooligosaccharide, xylooligosaccharide and malto-oligosaccharide, a third WSDF formed by pectin, β-glucan and polydextrose, a fourth WSDF formed by sodium alginate, gum arabic and resistant dextrin, and a fifth WSDF formed by xanthan gum, tragacanth gum and inulin; and
a mass ratio of three components within each of the first WSDF, the second WSDF, the third WSDF, the fourth WSDF or the fifth WSDF is (1-3):(1-3):(1-3).

3. A pharmaceutical composition that promotes metabolism of ellagic acid (EA) into urolithin A, comprising the WSDF according to claim 1 and EA; wherein
the WSDF and EA have a mass ratio of (100-250):3.

4. The pharmaceutical composition according to claim 3, wherein the WSDF and EA have a mass ratio of 250:3.

5. The pharmaceutical composition according to claim 3, wherein EA has a structure represented by formula I:

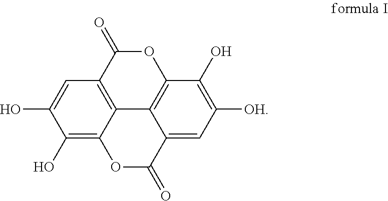 [SMILES: O=c1oc2c(O)c(O)cc3c(=O)oc4c(O)c(O)cc1c4c23]

6. A method for promoting metabolism of ellagic acid into urolithin A, comprising administering to a subject in need thereof the WSDF according to claim 1.

7. A product that promotes metabolism of EA into urolithin A, comprising the WSDF according to claim 1 as an active ingredient, and excipients.

8. The product according to claim 7, wherein the product that promotes metabolism of EA into urolithin A comprises at least one selected from the group consisting of a medicament, a health care product and a health food.

9. A method for preventing or treating a disease selected from the group consisting of diabetes, obesity, senescence and central nervous system lesions, comprising administering to a subject in need thereof a therapeutically effective amount of the pharmaceutical composition according to claim 3.

10. The method according to claim 9, wherein patients with the disease belong to one selected from the group consisting of metabotypes A, B and 0 populations of EA metabolism.

11. The pharmaceutical composition according to claim 3, wherein the WSDF comprises at least one selected from the group consisting of a first WSDF formed by gellan gum, guar gum and carrageenan, a second WSDF formed by fructooligosaccharide, xylooligosaccharide and malto-oligosaccharide, a third WSDF formed by pectin, β-glucan and polydextrose, a fourth WSDF formed by sodium alginate, gum arabic and resistant dextrin, and a fifth WSDF formed by xanthan gum, tragacanth gum and inulin; and
a mass ratio of three components within each of the first WSDF, the second WSDF, the third WSDF, the fourth WSDF or the fifth WSDF is (1-3):(1-3):(1-3).

12. The pharmaceutical composition according to claim 5, wherein the WSDF and EA have a mass ratio of 250:3.

13. The method according to claim 6, wherein the WSDF comprises at least one selected from the group consisting of a first WSDF formed by gellan gum, guar gum and carrageenan, a second WSDF formed by fructooligosaccharide, xylooligosaccharide and malto-oligosaccharide, a third WSDF formed by pectin, β-glucan and polydextrose, a fourth WSDF formed by sodium alginate, gum arabic and resistant dextrin, and a fifth WSDF formed by xanthan gum, tragacanth gum and inulin; and
a mass ratio of three components within each of the first WSDF, the second WSDF, the third WSDF, the fourth WSDF or the fifth WSDF is (1-3):(1-3):(1-3).

14. A method for promoting metabolism of ellagic acid into urolithin A, comprising administering to a subject in need thereof the pharmaceutical composition according to claim 3.

15. The method according to claim 14, wherein the WSDF and EA have a mass ratio of 250:3.

16. The method according to claim 14, wherein EA has a structure represented by formula I:

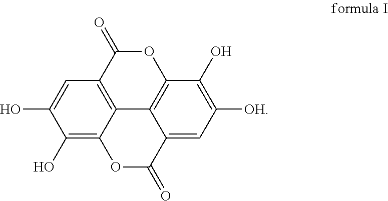 [SMILES: O=c1oc2c(O)c(O)cc3c(=O)oc4c(O)c(O)cc1c4c23]

17. The method according to claim 9, wherein the WSDF and EA have a mass ratio of 250:3.

18. The method according to claim 9, wherein EA has a structure represented by formula I: 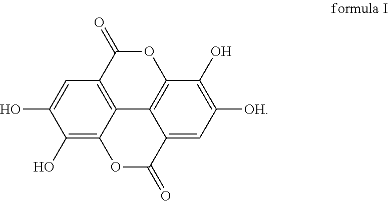 [SMILES: O=c1oc2c(O)c(O)cc3c(=O)oc4c(O)c(O)cc1c4c23]
